(19) United States
(12) Patent Application Publication
(10) Pub. No.: US 2015/0110750 A1
(43) Pub. Date: Apr. 23, 2015

(54) METHODS FOR USING AUTOLOGOUS FIBROBLASTS TO ALTER SKIN IDENTITY

(71) Applicants: THE JOHNS HOPKINS UNIVERSITY, Baltimore, MD (US); The Government of the United States of America, as represented by the Secretary of the Army, Fort Detrick, MD (US)

(72) Inventors: Luis Andres Garza, Baltimore, MD (US); Sewon Kang, Phoenix, MD (US); Jon H. Meyerle, Chevy Chase, MD (US)

(73) Assignees: The John Hopkins University, Baltimore, MD (US); The Government of the United States of America, as represented by the Secretary of the Army, Fort Detrick, MD (US)

(21) Appl. No.: 14/397,877

(22) PCT Filed: Apr. 30, 2013
(86) PCT No.: PCT/US2013/038914

§ 371 Date: Oct. 30, 2014

Related U.S. Application Data
(60) Provisional application No. 61/640,041, filed on Apr. 30, 2012.

(51) Int. Cl. A61K 35/12; C12N 5/071

(52) U.S. Cl. CPC A61K 35/33; C12N 5/0625
USPC 424/93.7

(57) ABSTRACT

The present invention relates to the field of autologous fibroblasts. More specifically, the present invention provides methods and compositions comprising autologous fibroblasts and uses thereof to alter skin identity. In certain embodiments, volar fibroblasts can be expanded for the ability to induce volar skin at the stump site in amputees. In other embodiments, fibroblasts from haired scalp can be expanded to ameliorate alopecias.

### CROSS-REFERENCE TO RELATED APPLICATIONS

This application claims the benefit of U.S. Provisional Application No. 61/640,041, filed Apr. 30, 2012, which is incorporated herein by reference in its entirety.

### STATEMENT OF GOVERNMENTAL INTEREST

This invention was made with U.S. government support under grant no. 1K08AR05566601 A1. The U.S. government has certain rights in the invention.

### FIELD OF THE INVENTION

The present invention relates to the field of autologous fibroblasts. More specifically, the present invention provides methods and compositions comprising autologous fibroblasts and uses thereof to alter skin identity.

### BACKGROUND OF THE INVENTION

A major effort for regenerative medicine is to change tissue identity. In dermatology, the applications of changing skin identity are wide and include reverting scars back to their original tissue type. These efforts will likely involve cellular therapy, as has already been done for example in injections of allogeneic fibroblasts to treat inherited bullous diseases. Although allogeneic cells are already routinely used in wound therapies, optimal results are impeded by a lack of knowledge regarding effective delivery and engraftment of stem cells to skin. Also, optimization of cellular therapy for chronic wounds or bullous diseases is difficult given the clinical variability and rarity of these conditions.

### SUMMARY OF THE INVENTION

The present invention is based, at least in part, on the discovery that site-specific autologous fibroblasts can be used to alter skin identify. In certain embodiments, volar fibroblasts can be expanded for the ability to induce volar skin at the stump site in amputees. In other embodiments, fibroblasts from haired scalp can be expanded to ameliorate alopecias.

Accordingly, in one aspect, the present invention provides methods and compositions useful for altering skin identity. In one embodiment, a method for altering skin identity in a patient comprises the step of transplanting autologous fibroblasts into the target skin site of a patient, wherein the autologous fibroblasts are obtained via a skin biopsy from the desired skin type site. In a specific embodiment, the target skin site is the stump site skin of an amputee and the desired skin type site is volar skin of the amputee. In another embodiment, the target skin site is an alopecia site on the patient and the desired skin type site is haired scalp. In an alternative embodiment, the target skin site is a scar and the desired skin type site is from an area adjacent to or surrounding the scar. In a further embodiment, the target skin site is discolored skin and the desired skin type is from an area adjacent to or surrounding the discolored skin. In a specific embodiment, the discolored skin is a port wine stain.

In another embodiment, the target skin site is a mismatched split thickness skin graft or other autologous skin graft and the desired skin type is from the contralateral skin of desired identity. Alternatively, the target skin site is a site with a predilection for a rash or ulcer and the desired skin type is normally resistant to that rash or ulcer. More specifically, the site with a predilection for a rash or ulcer is a pressure ulcer of the sacrum and the desired skin type that is normally resistant to that rash or ulcer is volar skin.

The present invention also provides a method for altering skin identify in a patient comprising the steps of (a) obtaining a tissue sample from the desired skin type site of the patient; (b) culturing the tissue to expand the fibroblasts; and (c) transplanting the expanded autologous fibroblasts into the target skin site of the patient. In a specific embodiment, the target skin site is the stump site skin of an amputee and the desired skin type site is volar skin of the amputee. In another embodiment, the target skin site is an alopecia site on the patient and the desired skin type site is haired scalp. Alternatively, the target skin site is a scar and the desired skin type site is from an area adjacent to or surrounding the scar. In yet another embodiment, the target skin site is discolored skin and the desired skin type is from an area adjacent to or surrounding the discolored skin. More specifically, in another embodiment, the discolored skin is a port wine stain.

In another specific embodiment of the present invention, a method for inducing volar skin at the stump site of amputees comprises the step of transplanting autologous volar fibroblasts to the non-volar stump site of the amputee. In certain embodiments, the transplantation step comprises injection of the autologous volar fibroblasts.

The present invention also provides a method for inducing volar skin at the stump site of amputees comprising the steps of (a) obtaining a volar skin biopsy from the amputee; (b) culturing the biopsy to expand the fibroblasts; and (c) transplanting the expanded autologous fibroblasts into the stump site of the amputee. In another embodiment, a method for treating alopecia in a patient comprises the step of transplanting autologous fibroblasts into the alopecia site of the patient, wherein the autologous fibroblasts are obtained via a skin biopsy from the haired scalp of the patient. In another embodiment, a method for treating alopecia in a patient comprises the steps of (a) obtaining a tissue sample from the haired scalp of the patient; (b) culturing the tissue to expand the fibroblasts; and (c) transplanting the expanded autologous fibroblasts into the alopecia site of the patient.

The present invention further provides a method for altering the skin identity of a scar in a patient comprising the step of transplanting autologous fibroblasts into the scar site of the patient, wherein the autologous fibroblasts are obtained via a skin biopsy from a skin site adjacent to or surrounding the scar site of the patient. In another embodiment, a method for altering the skin identity of a scar in a patient comprises the steps of (a) obtaining a tissue sample from a skin site adjacent to or surrounding the scar site of the patient; (b) culturing the tissue to expand the fibroblasts; and (c) transplanting the expanded autologous fibroblasts into the scar site of the patient.

In another aspect, the present invention provides pharmaceutical compositions useful for altering skin identity. In certain embodiments, a pharmaceutical composition comprises autologous fibroblasts for implantation. The present invention also provides autologous fibroblasts for use in a method of altering skin identity in a patient, wherein the autologous fibroblasts are obtained via a skin biopsy from the desired skin type site and are transplanted to into the target skin site of the patient. In the autologous fibroblasts for such use (a) the target skin site is the stump site skin of an amputee and the desired skin type site is volar skin of the amputee; (b) the target skin site is an alopecia site on the patient and the desired skin type site is haired scalp; (c) the target skin site is a scar and the desired skin type site is from an area adjacent to or surrounding the scar; or (d) the target skin site is discolored skin and the desired skin type is from an area adjacent to or surrounding the discolored skin.

More specifically, the pharmaceutical compositions comprise about 0.1 to about 9.9×10¹⁰ cells/ml, about 0.5 to about 9×10¹⁰ cells/ml, about 1 to about 9×10⁹ cells/ml, about 1 to about 8×10⁸ cells/ml, about 1 to about 7×10⁷ cells/ml, about 1 to about 6×10⁶ cells/ml, about 1 to about 5×10⁵ cells/ml. In more particular embodiments, the appropriate number of cells includes about 1 to about 9×10⁷ cells/ml, about 1 to about 8×10⁷ cells/ml, about 1 to about 7×10⁷ cells/ml, about 1 to about 6×10⁷ cells/ml, about 1 to about 5×10⁷ cells/ml, about 1 to about 4×10⁷ cells/ml, about 1 to about 3×10⁷ cells/ml, and about 1 to about 2×10⁷ cells/ml. Alternatively, the target amount can be adjusted within the formulation range to accommodate different indication doses.

The autologous fibroblast composition may comprise at least about 80%, at least about 85%, at least about 88%, at least about 89%, and at least about 90% fibroblasts. More specifically, the composition may comprise at least about 91%, at least about 92%, at least about 93%, at least about 94%, at least about 95%, at least about 96%, at least about 97%, at least about 98%, or at least about 99% fibroblasts.

The pharmaceutical compositions of the present invention may be administered by any effective route of administration. In specific embodiments, the administration route is by injection. In certain embodiments, the composition is administered through a sub-epidermal injection (intradermis) very close to the epidermal/dermal junction.

### BRIEF DESCRIPTION OF THE FIGURES

FIG. 1: Schematic outlining the creation of ectopic volar skin at the stump site of amputees.

FIG. 2: Image depicting palmoplantar keratodermas in a patient with KRT9 mutation.

FIG. 3: Normal expression of KRT9 (n=3, p<0.05).

FIG. 4: Volar thawed fibroblasts (1 million) induce ectopic KRT9 expression in foreskin keratinocytes (0.25 million) after 3 days of co-culture in 10 cm plates (n=3, p<0.05; In vitro 2-D co-culture assay).

FIG. 5: In vitro 3-D assay demonstrating epidermal and stratum corneum thickening of foreskin keratinocytes (1 million) with thawed volar fibroblasts (sole vs. dorsum of foot; 1 million in rat tail collagen type I) in 4.2 cm² insert at air interface for 3 weeks (20×).

FIG. 6: HoxA13 is necessary for KRT9 induction in thawed fibroblasts. (n=3, p<0.05). Fibroblasts nucleofected (Lonza 4D) with 12 pmoles of siRNA (Dharmacon #3209) prior to co-culture; verified to be >50% knockdown.

FIG. 7: LMX1b expression inversely reflects KRT9 induction ability in passaged fibroblasts as measured by taqman ABI qRT-PCR (n=3, p<0.05).

FIG. 8: LMX1b knockdown increases DKK-1 at day 3 post knockdown (n=3, p<0.05).

FIG. 9: LMX1b knockdown endows the dorsum of the foot thawed fibroblasts with new capacity to induce KRT9 (n=3. p<0.05).

FIG. 10: Diverse fibroblasts acquire the ability to induce KRT9 comparing scrambled to LMX1b knockdown (n=3, p<0.05).

FIG. 11: Confirmation of arrays: PAX9 is a volar homeobox gene in fibroblasts (n=3, p<0.05).

FIG. 12: LMX1b knockdown induces PAX9 in fibroblasts alone (n=3, p<0.05).

FIG. 13: hSHOX mouse homologue is suppressed in LMX1b knockout mouse limb bud tissue.

FIG. 14: EMX2 is suppressed in LMX1b knockout mouse limb bud tissue.

FIG. 15: 24 hrs after GFP plasmid transient transfections performed on 1 million non-volar fibroblasts with 2 μg of pMAX plasmid by Lonza 4-D nucleofection.

FIG. 16: “HAT” assay for skin reconstitution reflects the identity of added fibroblasts. A slurry of 10 million single cell neonatal mouse keratinocytes alone (top) or with 10 million neonatal mouse dermal fibroblasts (bottom) were injected into a 1 cm diameter silicon “HAT” chamber at the back of a NUDE mouse and harvested after 4 weeks when morphogenesis is complete.

FIG. 17: Sole fibroblasts proliferate more slowly at later passages (n=3, p<0.01). Sole and Foot fibroblasts were seeded at 0.5 million cells in a 60 cm dish and allowed to expand for 7 days before cell counting.

FIG. 18: In vivo Optical Coherence Tomography (OCT; VivoSight) of human volunteer demonstrates thicker (0.11 vs. 0.31 mm) epidermis in volar skin.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

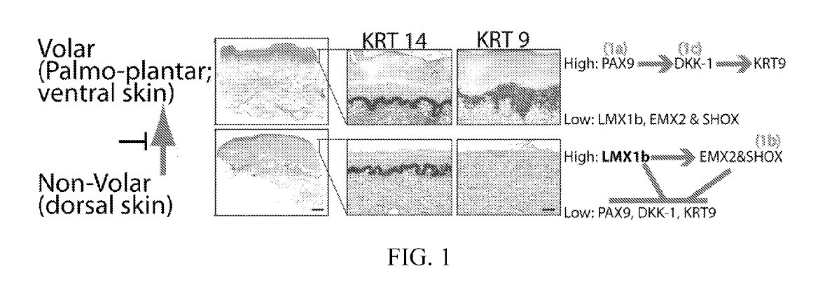

In certain embodiments, the present invention is applicable to the enhancement of prosthetic use in amputees. In the United States, it is estimated that by 2050, 3.6 million people will have lost a limb. While dramatic advancements are being made in prosthetic design, they are all limited by skin-breakdown at the stump site. In a recent review we conducted, 48% of Vietnam veterans for example still have skin problems more than 40 years after their amputation. As described herein, in certain embodiments, the methods of the present invention convert the identity of the skin at the stump site to volar (palmo-plantar) skin to enhance friction and irritant resistance (FIG. 1). In the same way people do not develop skin breakdown at the soles of their feet, volar skin at the stump site should enhance prosthetic use.

In other embodiments, the present invention utilizes site specific autologous fibroblasts to alter tissue identity. For example, fibroblasts from haired scalp can be utilized to treat alopecias. In other embodiments, site-specific fibroblasts can be utilized to treat scar tissue, altering the scar tissue identity to better match adjacent and surrounding skin. Further, site-specific fibroblasts can be utilized to alter burn scar tissue identity.

The present invention is based on a concept in developmental biology, namely, the mesenchymal (dermal fibroblast) control of epithelial (keratinocyte) function. Early experiments swapping chicken and duck epithelium and mesenchyme established that mesenchyme controls epidermal identity. The present invention translates this fundamental concept to help improve the quality of life for amputation victims.

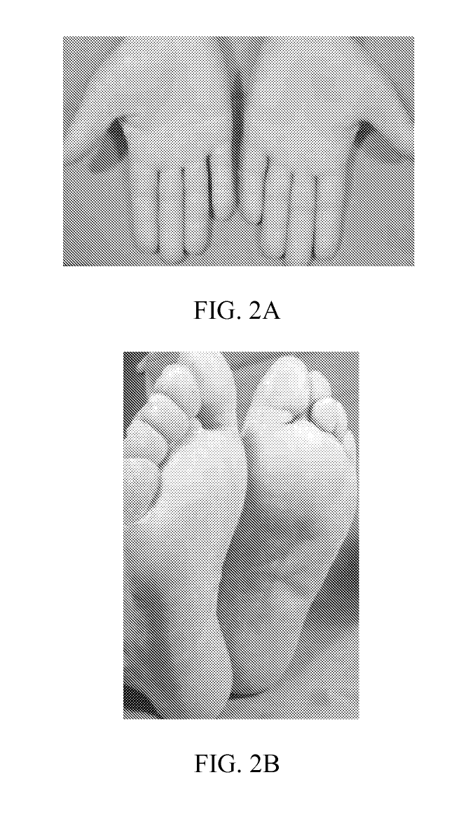

Volar keratinocytes express KRT9, a keratin which is responsible for structural resiliency. KRT9 is mutated in select cases of Epidermolytic Palmoplantar Keratoderma (EPK; OMIM 144200). These patients have only palmo/plantar symptoms which confirms the limited expression of KRT9. KRT9 is an ideal read-out of volar function also because it provides integral structural support to volar skin, as evidenced by EPK patient's symptoms of compensatory thickened palms and soles (FIG. 2). Evidence of KRT9's structural function is also its normal expression pattern in healthy individuals. KRT9 is specifically upregulated at the points of highest compressive stress of the skin, the top of the papillary ridges. No other gene is accepted to be limited to volar skin.

Volar skin has thickened stratum corneum and epidermis. Early experiments using human cells confirmed that dermal fibroblasts control KRT9 expression. It was shown that human plantar fibroblasts can convert adult trunk keratinocytes into a thickened KRT9 positive epidermis when transplanted to SCID mice. Also, when adult human non-volar epidermis is transplanted to palm dermis, the epidermis thickens and expresses KRT9, indicating that palmar dermis directs epidermal identity. The present invention establishes the reciprocal concept: fibroblasts are capable of inducing KRT9 and can induce ectopic adult volar skin in humans.

Further early work demonstrated that DKK-1 is elevated in volar fibroblasts and has the ability itself to increase epidermal thickness and decrease pigmentation in volar epidermis. Similarly, distal homeobox A13 (HOXA13) was shown to be necessary for KRT9 induction and induces Wnt5a which itself also can stimulate KRT9 expression. In addition, it was shown that fibroblast homeobox gene expression signatures are maintained in culture. However, HOXA13 specified proximal-distal fate, and was not a specific homeobox controlling volar skin identity. Therefore, the present invention also aims to identify overlapping genes of palms and soles. Indeed, the present inventors discovered that the homeobox gene LMX1b specifically regulates volar skin identity, in that its suppression in a diverse array of fibroblasts allows for the novel ability to induce KRT9.

As described herein, the present inventors, in the search for therapies to induce ectopic volar skin, focused on the fibroblasts themselves—which maintain their identity in culture. Fibroblasts are specialized cells in the skin that produce collagen and other extracellular matrix components. They are the cells from which connective tissues develop and, as such, play critical roles in the development of human tissue, including the ability to synthesize extracellular matrix components that contribute to skin texture and the secretion of matrix fibers, including collagen.

In certain embodiments, an autologous fibroblast product is provided. The cell therapy product comprises a suspension of autologous fibroblasts, grown from a biopsy of each individual's own skin using standard tissue culture procedures. Fibroblasts isolated from the tissue via enzymatic digestion are expanded to a quantity sufficient for injection into the patient's target treatment area.

The autologous fibroblasts are derived from a biopsy of the recipient's own skin followed by expansion in culture using standard cell culture techniques. Skin tissue (dermis and epidermis layers) is biopsied from a subject's relevant area. In one embodiment, the starting material comprises a three 3-mm or four 4-mm punch skin biopsies collected using standard aseptic practices. The biopsies are collected by the treating physician, placed into a vial containing sterile phosphate buffered saline (PBS) or other transport media. The biopsies are cultured and expanded for use in the methods described herein. Alternatively, the biopsies can be shipped in a 2-8° C. refrigerated shipper to a facility for culture, expansion and/or storage.

In particular embodiments, the active component of this therapy is autologous cultured fibroblasts. As another example, the fibroblasts are cultured, using standard methodologies, from a 4-mm punch biopsy that includes the epidermal and dermal layer taken from a volar (palm or sole) or a non-volar skin site. During and after in vitro expansion, the fibroblasts are harvested and quality control tests are performed. Greater than about 95% of the final suspension comprises fibroblasts as most of the keratinocytes are removed in the initial stages of processing. Once the final suspension reaches at least about 37.5 million fibroblasts, the cell suspension is cryopreserved in freezing medium containing, for example, human serum albumin, hetastarch and dimethyl sulfoxide (DMSO) (2.5% human serum albumin, 5% DMSO, 6% hetastarch in sterile saline) at a defined fibroblast cell concentration. Before clinical use, cells are thawed, and then injected via an intradermal route into the autologous graft site within 24 hours. In one specific embodiment, the transplant volume comprises approximately 37.5 million cells in 0.75 mL of freezing media.

In one embodiment, autologous fibroblasts may be prepared as follows:

Biospy Processing.

Following biopsy, the tissue sample is inspected and washed prior to enzymatic digestion. After washing, in certain embodiments, a Liberase Digestive Enzyme Solution can be added without mincing, and the biopsy tissue is incubated at 37.0±2° C. for about one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Helidelburg, Germany). After digestion, growth media (e.g., IMDM, GA, 10% Fetal Bovine Serum (FBS)) is added to neutralize the enzyme, cells are pelleted by centrifugation and resuspended in 5.0 mL growth media. Any suitable growth media can be used. Alternatively, centrifugation is not performed, with full inactivation of the enzyme occurring by the addition of growth media only.

Fibroblast Seeding.

Growth media is added prior to seeding of the cell suspension into a T-175 cell culture flask for initiation of cell growth and expansion. A T-75, T-150, T-185 or T-225 flask can be used in place of the T-75 flask. Cells are incubated at 37±2.0° C. with 5.0±1.0% CO₂ and fed with fresh growth media about every three to five days. All feeds in the process are performed by removing half of the growth media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting.

Fibroblast Expansion.

When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask.

Morphology is evaluated at each passage and culture purity is monitored throughout the process prior to harvest. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies.

In certain embodiments, cells are incubated at 37±2.0° C. with 5.0±1.0% CO2 and fed every three to five days in the T-500 flask and every five to seven days in the ten layer cell stack (10CS). Cells should not remain in the T-500 flask for more than 10 days prior to passaging. Quality Control (QC) release testing for safety includes sterility and endotoxin testing. When cell confluence in the T-500 flask is ≧95%, cells are passaged to a 10 CS culture vessel. Alternately, two Five Layer Cell Stacks (5 CS) or a 10 Layer Cell Factory (10 CF) can be used in place of the 10 CS. 10CS. Passage to the 10 CS is performed by removing the spent media, washing the cells, and treating with Trypsin-EDTA to release adherent cells in the flask into the solution. Cells are then transferred to the 10 CS. Additional growth media is added to neutralize the trypsin and the cells from the T-500 flask are pipetted into a 2 L bottle containing fresh growth media. The contents of the 2 L bottle are transferred into the 10 CS and seeded across all layers. Cells are then incubated at 37±2.0° C. with 5.0±1.0% CO₂ and fed with fresh growth media every five to seven days. Cells should not remain in the 10CS for more than 20 days prior to passaging.

Cell Harvest.

When cell confluence in the 10 CS is 95% or more, cells are harvested. Harvesting is performed by removing the spent media, washing the cells, treating with Trypsin-EDTA to release adherent cells into the solution, and adding additional growth media to neutralize the trypsin. Cells are collected by centrifugation, resuspended, and in-process QC testing performed to determine total viable cell count and cell viability.

If additional cells are required after receiving cell count results from the primary 10 CS harvest, an additional passage into multiple cell stacks (up to four 10 CS) can be performed. For additional passaging, cells from the primary harvest are added to a 2 L media bottle containing fresh growth media. Resuspended cells are added to multiple cell stacks and incubated at 37±2.0° C. with 5.0±1.0% CO₂. The cell stacks are fed and harvested as described above, except cell confluence must be 80% or higher prior to cell harvest. The harvest procedure is the same as described for the primary harvest above. A mycoplasma sample from cells and spent media is collected, and cell count and viability performed as described for the primary harvest above.

In particular embodiments, the passaged dermal fibroblasts are rendered substantially free of immunogenic proteins present in the culture medium by incubating the expanded fibroblasts for a period of time in protein free medium. The method decreases or eliminates immunogenic proteins be avoiding their introduction from animal-sourced reagents. To reduce process residuals, cells can be cryopreserved in protein-free freeze media, then thawed and washed prior to prepping the final injection to further reduce remaining residuals.

Cryopreservation of Cells.

Accordingly, in certain embodiments, the composition comprises a population of viable, autologous human fibroblast cells suspended in a cryopreservation medium comprising Iscove's Modified Dulbecco's Medium (IMDM) and Profreeze-CDM™ (Lonza, Walkerville, Md.) plus 7.5% dimethyl sulfoxide (DMSO). Alternatively, a lower DMSO concentration may be used in place of 7.5% or CryoStor™ CS5 or CryoStor™ CS10 (BioLife Solutions, Bothell, Wash.) may be used in place of IMDM/Profreeze/DMSO.

In another embodiment, autologous fibroblasts may be prepared as follows:

Biospy Processing.

The processed biopsies are prepared for tissue culture in a biological safety cabinet (BSC) Level I using all sterile (autoclaved or individually packaged) instruments. The biopsy is rinsed twice by dipping in 25 mL of sterile rinse buffer containing Dulbecco's Phosphate Buffered Saline or PBS (Sigma D8537) supplemented with ABX Antibiotic Antimycotic Solution (100×), Stabilized (Sigma A5955) at a 1× final concentration. Each milliliter of the 100× antibiotic/antimycotic solution contains 10,000 units of penicillin, 10 mg of streptomycin, and 25 μg of amphotericin B. After rinsing the biopsied skin in antibiotic/antimycotic solution, it is placed in a 50-mL conical tube (Becton Dickinson) containing 25 mL of PBS (with 1ABX) wash buffer and subjected to two washes. In the first wash, the tube is shaken vigorously and the skin is allowed to soak for a few minutes. Using a pair of forceps sterilized with 70% ethanol, the skin is transferred to another 50-mL conical tube containing PBS/ABX wash buffer. In the second wash, the tube again is shaken vigorously. The skin may begin to float. The skin is allowed to soak for a few minutes in this final wash.

After washing, the skin pieces are removed from the conical tube and transferred to sterile 10 cm culture dishes. The standard operating procedure requires the operator to visualize the top layer epidermis and bottom layers comprising of the dermis and subcutaneous fat. The subcutaneous fat is removed using a sterile scalpel blade and each separated piece is bisected.

Each skin biopsy piece is then transferred using a pair of sterile forceps to a sterile 15-mL conical tube (Becton Dickinson) containing 10 mL of fibroblast media. The fibroblast medium consists of Chemically Defined FGM-CD fibroblast growth medium (Lonza CC-3132, composed of CC-4126 and CC-3131) supplemented with high quality defined Fetal Bovine Serum (Invitrogen 16000-044) to a 2% final concentration, as well as 2000 μL of Dispase I (BD Biosciences catalog #354235) and a 1× final concentration of ABX antibiotic/antimycotic solution.

The tubes are tightly closed and incubated overnight in the refrigerator (4° C.) without shaking After 12 hours the tubes are shaken gently and returned to the refrigerator. Epidermis will separate out in approximately 18 hours of Dispase I treatment. After 18 hours of incubation at 4° C. tubes are removed from refrigeration and transferred to the biosafety cabinet. Before unscrewing the caps tubes are disinfected with 70% ethanol. Each piece of the biopsy is removed from the fibroblast media and placed on a sterile, dry 10-cm culture dish (Fisher Scientific) using autoclaved forceps. No dispase is to contact the tissue or cells in any subsequent step. Each piece is laid out on the culture dish and aerated for 2-5 minutes inside a sterile safety cabinet to enable attachment of the dermis to the dish. After aeration the layers are carefully peeled apart using two pairs of sterile forceps (starting from the edges) by holding the dermis with one pair of forceps and the epidermis with another.

Once separated, each dermis piece is placed in one well of a sterile uncoated 6-well tissue culture plate (Fisher Scientific) with the “epidermal” side facing down and spread to a single layer. Each culture plate should contain only one dermis. The lid of the culture plate is replaced and the plate is transferred to a sterile 37° C. incubator. The lid is removed and the dermis is allowed to aerate in the incubator for approximately 15-20 minutes enabling the dermis to stick to the bottom of the well. All fibroblast expansion from the isolated dermis is done in fibroblast media that does not contain dispase.

Fibroblast Seeding.

Following aeration of the skin pieces, 2 mL of fibroblast medium is carefully added to the well containing the dermis piece making sure to not disturb the dermis. This is accomplished by tilting the dish slightly so the medium is added slowly from the edge rather than directly onto the dermis. The culture plate is slowly returned to the horizontal position and transferred to a sterile 37° C., CO₂ incubator. The plate is incubated undisturbed for 2-3 days. For example, if the dermis is processed on Friday, 2 mL of fresh fibroblast medium is added and the plate is left in the incubator until Monday afternoon. Culture medium is changed three times per week, noting the color of medium at each change. If the culture turns yellow the media should be changed more frequently, e.g. every other day. At every culture change the cells are visually inspected under an inverted microscope for presence of infection, i.e., presence of filaments, increased turbidity, or particulate matter or motile organisms. The culture is also observed for status of fibroblast expansion. Fibroblasts should begin to grow out of the dermis in about eight days. The culture supernatant is aspirated off using a sterile pipette and automatic pipettor.

Fibroblast Expansion.

Fibroblast culture confluency is checked routinely and estimated based on the amount of free space on the well. In order to maintain confluency, 30-80% of the culture is split into new vessels as needed. If the confluency is greater than 80%, the culture is split and the fibroblast cells are sub-cultured as necessary to expand the cultures. For sub-culturing, cells are incubated in media supplemented with Trypsin LE-express (Invitrogen 12605-010) for minimal amount of minutes before cells lift off the plate (typically less than 5 minutes).

For the first passage, cells in each well are washed twice with 4 mL each of sterile PBS. Two mL of trypsin are added to the dish and incubated for five minutes or until cells lift off the dish. One mL of sterile FBS is added to the well to neutralize the trypsin. Unwashed cells are plated after trypsinization. Ten μL of the trypsin inactivated solution are added to 5 μL of 0.4% trypan blue and loaded on a Countess cartridge. The total number of cells is determined using a Countess automated cell counter. Forty mL of sterile PBS (supplemented with antibiotic/antimycotic solution) is added to cells in a 50 mL conical tube. Cells are centrifuged at 200×g for 5 minutes, then resuspended in 12 mL of fibroblast media. The number of cells is recorded in the production log as described above. Two million cells are added to labeled 75 cm2 (T-75) tissue culture flasks (Fisher Scientific).

For subsequent passages, cells are washed with two aliquots of 20 mL each of sterile PBS followed by incubation of the culture in 5 mL of trypsin for 5 minutes or until cells lift off the flask. One milliliter of sterile FBS is added to the flask to neutralize the trypsin. Cells are transferred to a 50 mL conical tissue culture tube followed by addition of 40 mL of sterile PBS (supplemented with antibiotic/antimycotic solution). Cells are counted in a Countess automated cell counter as described above. Cells are centrifuged for 5 minutes at 200×g, and the pellet is resuspended in 12 mL of fibroblast media. Finally, the cells are added to a new labeled T-75 tissue culture flask. The seeding density will be 2 million cells per T-75.

For the final two passages, when the total cell number has reached 5 million cells, we subsequently maintain cells in the aforementioned media but the media will not contain any antibiotics or antimycotics. Therefore the media will be chemically Defined FGM-CD fibroblast growth medium (Lonza CC-3132) supplemented with high quality defined Fetal Bovine Serum (Invitrogen 16000-044) to a 2% final concentration.

Cell Harvest.

Cultured fibroblasts are harvested when the cellular density reaches 120% of the target cell number for injection. For example, for an injection of 37.5×10⁶ cells, 45×10⁶ cells are harvested. Before the final wash of cells, excess media will be saved for Mycoplasma testing. The cells are counted using the Countess automated cell counter. Once 45×10⁶ cells are isolated then an aliquot will be frozen. Three small aliquots containing approximately 1×10⁶ cells each are packaged for sterility, endotoxin, potency, and stability testing.

The culture supernatant is first inspected visually both grossly and under a tissue culture microscope to ensure no evidence of contamination. Then, the culture supernatant is aspirated off using a sterile pipette and automatic pipettor. Cultured fibroblasts are removed from the growing surface with trypsin following the procedure described above for passaging the cultures except that cell are processed for cryopreservation as described below.

Cryopreservation of Cells.

After trypsinization, the cell suspension is diluted with PBS and centrifuged at 200×g for 5 minutes in a Beckman Coulter centrifuge (Model #392187). Cells are resuspended in PBS and pelleted again. The final wash is aspirated off and the pellet is resuspended in 95% cryopreservation media (cryoprotectant) consisting of 65 mL of Hespan (6% Hetastarch) (B. Braun Medical, Inc.), and 6.5 mL of 25% Human Serum Albumin (HSA) (Gemini Bio-Products, catalog #800-126P).

Cells are resuspended at a density of 3-4×10⁷ cells per 0.75 mL of crypoprotectant per 2-mL cryovial (Nalgene Cryovial, catalog #5012-0020). The vial(s) are subjected to slow freezing for at least 24 hour at −80° C. in special room temperature isopropanol containers-Mr. Frosty-Nalgene (Sigma, catalog #C1562). In this simple process, vials are simply placed inside the Mr. Frosty and allowed to slowly cool by placing the room temperature Mr. Frosty container in a −80 freezer. Once the slow freezing is completed the vials are transferred to a liquid nitrogen freezer and stored in the vapor phase (−150° C.).

Autologous Fibroblast Preparations and Administration.

At the completion of culture expansion, the cells are harvested and washed, then formulated to contain an appropriate number of cells. Such pharmaceutical compositions comprising autologous fibroblasts include, but are not limited to about 0.1 to about 9.9×10¹⁰ cells/ml, about 0.5 to about 9×10¹⁰ cells/ml, about 1 to about 9×10⁹ cells/ml, about 1 to about 8×10⁸ cells/ml, about 1 to about 7×10⁷ cells/ml, about 1 to about 6×10⁶ cells/ml, about 1 to about 5×10⁵ cells/ml. In more particular embodiments, the appropriate number of cells includes about 1 to about 9×10⁷ cells/ml, about 1 to about 8×10⁷ cells/ml, about 1 to about 7×10⁷ cells/ml, about 1 to about 6×10⁷ cells/ml, about 1 to about 5×10⁷ cells/ml, about 1 to about 4×10⁷ cells/ml, about 1 to about 3×10⁷ cells/ml, and about 1 to about 2×10⁷ cells/ml. Alternatively, the target amount can be adjusted within the formulation range to accommodate different indication doses.

The autologous fibroblast composition may comprise at least about 80%, at least about 85%, at least about 88%, at least about 89%, and at least about 90% fibroblasts. More specifically, the composition may comprise at least about 91%, at least about 92%, at least about 93%, at least about 94%, at least about 95%, at least about 96%, at least about 97%, at least about 98%, or at least about 99% fibroblasts.

The pharmaceutical compositions of the present invention may be administered by any effective route of administration. In specific embodiments, the administration route is by injection. In certain embodiments, the composition is administered through a sub-epidermal injection (intradermis) very close to the epidermal/dermal junction.

Without further elaboration, it is believed that one skilled in the art, using the preceding description, can utilize the present invention to the fullest extent. The following examples are illustrative only, and not limiting of the remainder of the disclosure in any way whatsoever.

### Examples

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Robust System for Testing KRT9 Induction.

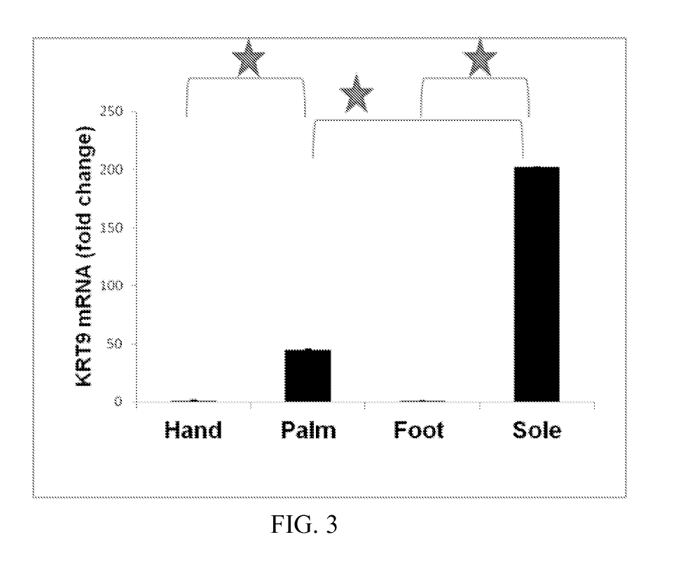

The present inventors are able to efficiently and rapidly test for the ability of fibroblasts to induce KRT9 expression in keratinocytes. In normal tissue, KRT9 is uniquely expressed in volar skin (FIG. 3). Interestingly, we found that among volar sites, the sole has higher levels of KRT9, probably because of greater pressure at that site. To try to replicate these findings, we initiated a robust in vitro system.

We first obtained human biopsies from both volar and nonvolar sites of the hands and feet (Hopkins IRB approved NA_{—}00033375) and expanded fibroblasts using conventional methods. Aliquoted fibroblasts (P2) were frozen for future use, then thawed and expanded. We expanded keratinocytes from foreskin excisions. Then using optimized protocols for the ideal ratio (1kers:4fibros), and time (3 days), we cultured these cells together in a conventional 2-D plate and harvest for qRT-PCR.

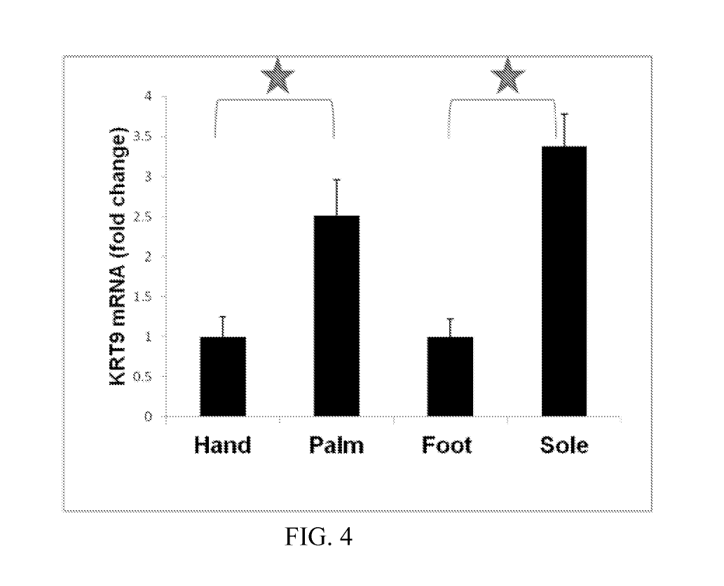

We found that volar fibroblasts have the capacity to induce KRT9 (FIG. 4). We found that they did so through passage 16, and non-volar cells never spontaneously did so. Normally KRT9 is expressed in KRT11+ suprabasal cells in vivo. Because the 2D system consists of basilar type keratin expression (K4+, K15+, KRT11−), the KRT9 induction levels are lower than in vivo. Another reason for the lower levels of KRT9 with in vitro model systems is the absence of pressure which is hypothesized to augment KRT9 expression. Within volar epidermis, KRT9 is most expressed at the center of papillary ridges that are thought to have the highest level of compressive stress. Nevertheless, our in vitro results also suggest a recapitulation of the normally higher amount of KRT9 induction by sole fibroblasts compared to palm fibroblasts (p=0.1).

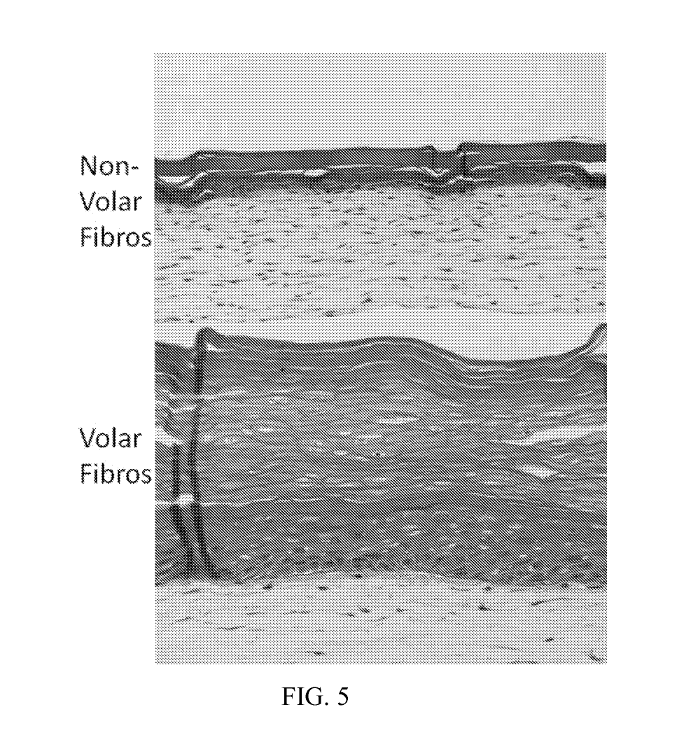

Our laboratory also initiated the use of 3-D cultures to assess how volar fibroblasts affect not just KRT9 expression, but more broad features of volar epidermis. The results show that the epidermis is thicker using volar fibroblasts (FIG. 5), similar to in vivo (FIG. 1). Also, KRT9 is induced by RT-PCR (17 fold).

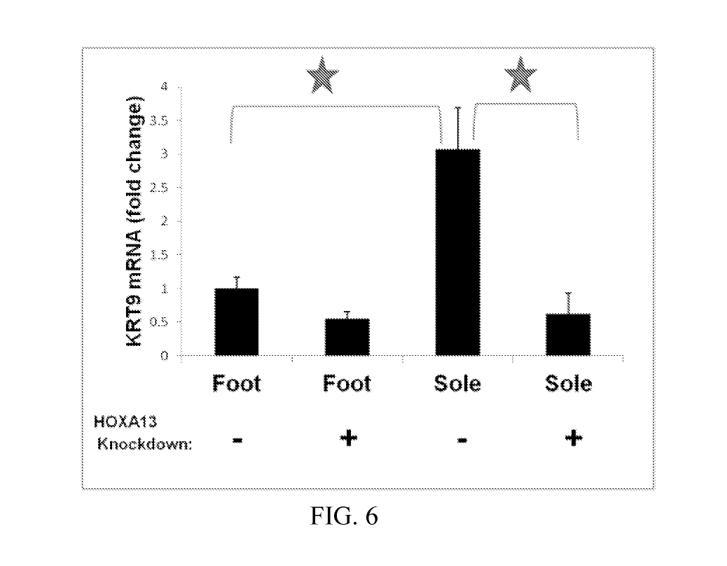

As additional validation for our system, we have confirmed the necessity of the distally active homeobox gene HoxA13 for the induction of KRT9. Expanded volar fibroblasts were treated with siRNA knockdown prior to co-culture with foreskin keratinocytes. While mock knockdown cells retain the ability to induce KRT9, HOXA13 deficient fibroblasts lose the ability (FIG. 6). While this experiment implies that HOXA13 is necessary for KRT9 induction, HOXA13 is unlikely to be involved specifically in the volar phenotype. Because HOXA13 is important for distal identity, it is likely only permissive for the volar phenotype. It has no described role in dorsal-ventral patterning and was not strongly associated with the volar skin gene signature (see below).

Microarray Characterization of Conserved Volar Gene Signature.

Despite the few published reports on the histology of volar skin, to our knowledge none describe the unique gene expression patterns. We therefore have performed exhaustive microarrays on human whole tissue (24), epidermis (24)/dermis (24), and cultured fibroblasts (24) to search for a conserved volar gene signature. For statistical analyses, we capitalized on the presence of 2 volar tissues (palms, soles) to search for common genes.

We first confirmed that KRT9 was the highest expressed gene in volar epidermis (of 44726 transcripts, p=0.0001). As cultured volar fibroblasts have the ability to faithfully induce KRT9 ectopically (FIG. 4), we focused on gene expression of these cells. Interestingly, homeobox gene mRNAs—centrally important in embryonic patterning—were still highly abundant in adult volar fibroblasts to presumably maintain their tissue identity (PAX9, SHOX, LMX1b, EMX2; Comparison to non-volar in Table 1). In comparison, signaling molecules published to promote KRT9 expression were comparatively lower expressed (Wnt5a, DKK1; Table 1). We therefore hypothesized that human adult skin tissue identity is actively maintained and that manipulations of these transcription factors might allow conversion of skin identity.

TABLE 1
Gene Chip Results for Normal Volar Fibroblast Gene Signature.

| | rank (of | | |
|---|---|---|---|
| name | 46,878) | fold | p value |

Genes up in volar fibroblasts

| | | | |
|---|---|---|---|
| PAX9 | 19 | 2 | 0.004 |
| WNT5a | 168 | 1.5 | 0.058 |
| DKK1 | 8396 | 1.2 | 0.744 |

Genes down in volar fibroblasts

| | | | |
|---|---|---|---|
| SHOX | 1 | −4 | 0.005 |
| LMX1b | 2 | −3.7 | 0.004 |
| EMX2 | 9 | −2.3 | 0.017 |

ANOVA analysis done for triplicate samples of human volar/non-volar fibroblast (P2) of both hands and feet for RNA Affymetrix Exon chip transcript abundance values.

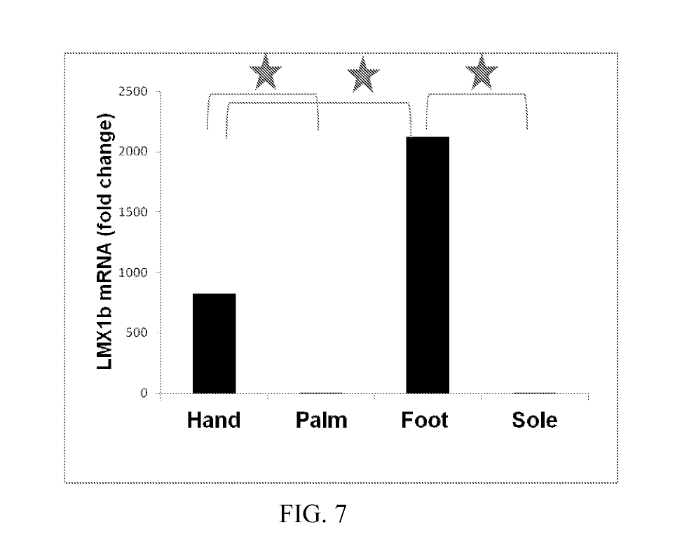

Based upon our microarray analysis, we focused on LMX1b. Heterozygous mutations result in Nail Patella syndrome, with defects on the dorsal nails and patellas (OMIM 161200). No homozygous mutations have been published in humans. However, homozygous knockout mice develop duplications of ventral structures. Postembryonic functions for LMX1b are unknown. Our hypothesis was that LMX1b might be a master suppressor of the volar phenotype—even active in adulthood. We confirmed LMX1b was almost completely suppressed in human volar whole dermis (data not shown). We then confirmed that LMX1b was suppressed in volar fibroblasts—and noted that LMX1b inversely correlated with KRT9 levels not only with respect to volar/nonvolar but also upper/lower extremity (FIG. 7).

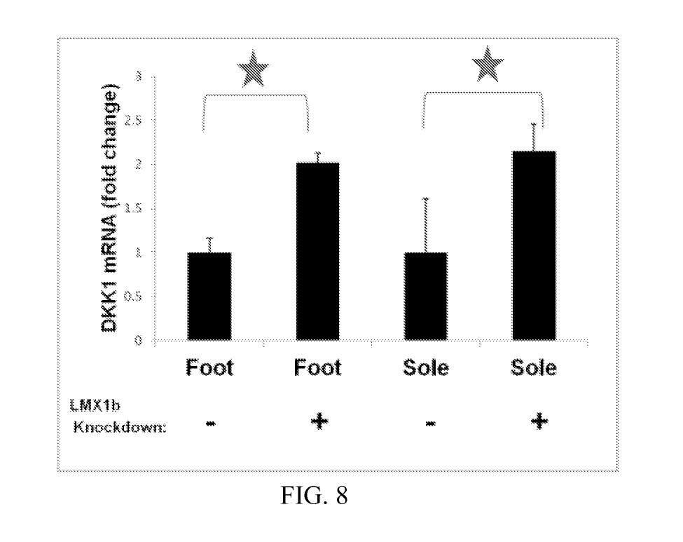

As a first step to determining LMX1b function, we next used siRNA treatment to knockdown LMX1b (80% reduction) to test whether it induced a signaling molecule known to promote the volar phenotype—DKK-1. We discovered that LMX1b knockdown indeed increased DKK-1 (FIG. 8) levels, suggesting that LMX1b might act as a repressor for volar phenotype in fibroblasts.

LMX1b is a Master Suppressor of the Volar Phenotype.

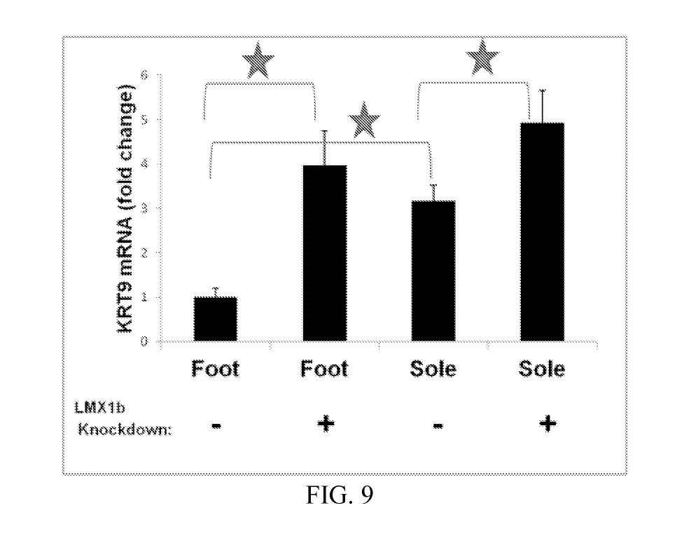

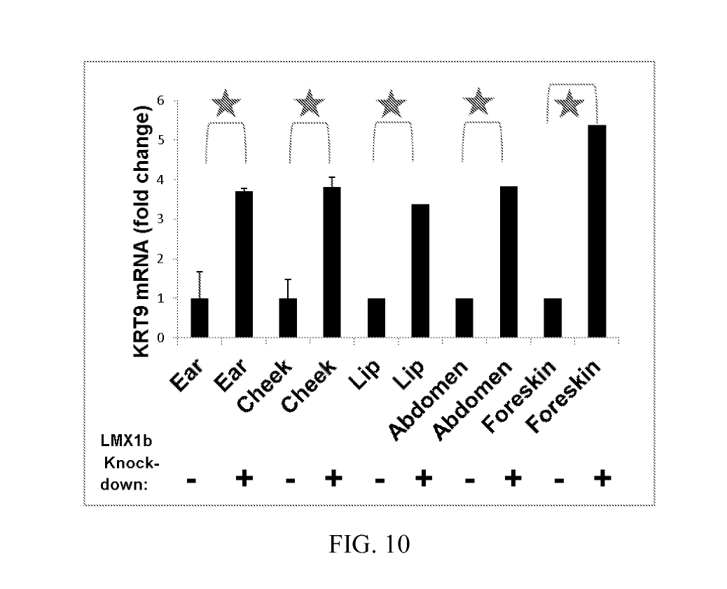

To probe LMX1b function, we then tested whether, under LMX1b knockdown, fibroblasts acquired the ability to induce KRT9. Indeed, as shown in FIG. 9 knockdown of LMX1b allowed non-volar fibroblasts from the dorsum of the foot to induce KRT9 in foreskin keratinocytes. We next tested if LMX1b knockdown could induce non-limb fibroblasts to stimulate KRT9 expression. We found that a diverse array of fibroblasts from foreskin, ear, cheek, lip and abdomen all acquired the ability to induce KRT9 with LMX1b knockdown (FIG. 10). These results clearly demonstrate that LMX1b suppresses a volar phenotype in a diverse collection of adult fibroblasts.

The ability of LMX1b knockdown in fibroblasts to induce KRT9 in non-volar keratinocytes gives a unique opportunity to dissect the necessary and sufficient pathways leading to the volar phenotype. To do so, we asked which genes follow three criteria: 1) genes highly enriched in palm fibroblasts relative to dorsum of the hands, 2) genes highly enriched in sole fibroblast relative to dorsum of the feet, and 3) genes which are highly enriched after LMX1b knockdown in diverse non-limb fibroblasts.

Candidate Downstream Genes from LMX1b.

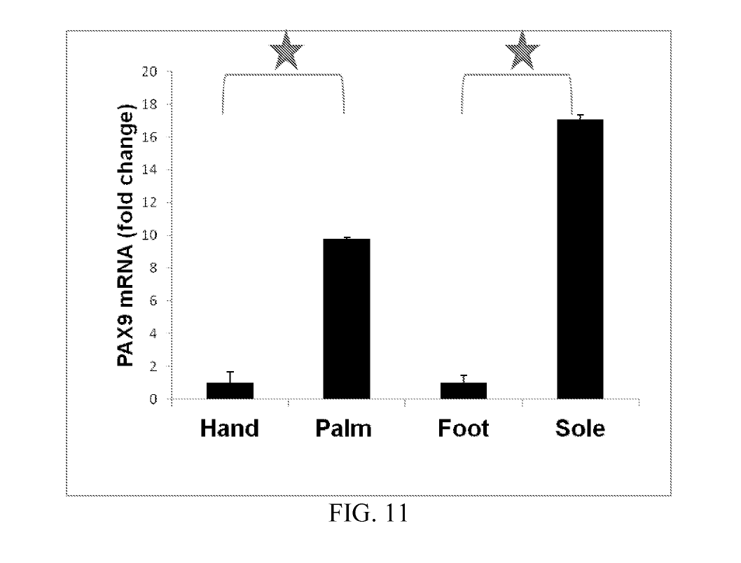

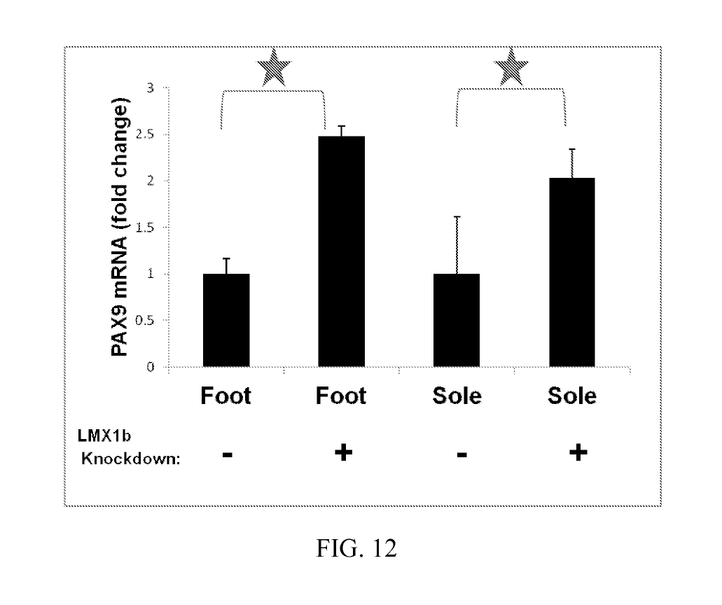

One of the primary candidate genes which fit these criteria is the homeobox gene PAX9. PAX9 is the transcription factor most enriched in volar tissues (Table 1). We confirmed the microarray by RT-PCR to verify that PAX9 is a volar homeobox gene (FIG. 11). Furthermore, knockdown of LMX1b in non-volar fibroblasts, results in significantly increased PAX9 gene expression (FIG. 12), consistent with results of LMX1b knockout mice. Finally, PAX9 null mice themselves show limb defects. These data suggest that PAX9 induction is downstream of LMX1b suppression.

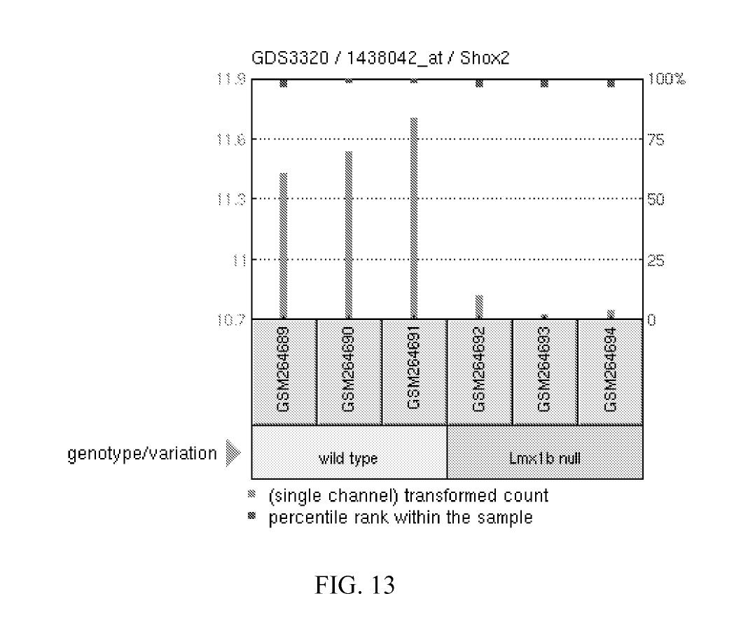

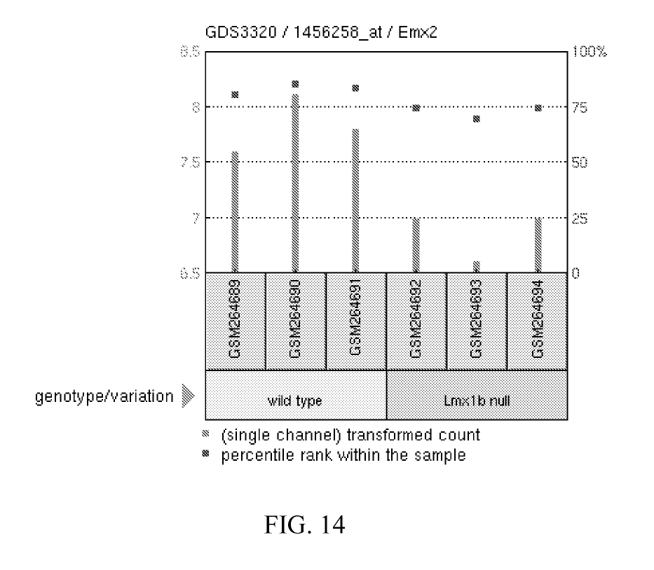

While PAX9 is upregulated in volar tissues, it is possible that other genes are downregulated upon LMX1b knockdown that are responsible for volar phenotype induction. The top genes which are suppressed in volar fibroblasts besides LMX1b are SHOX and EMX2 (Table 1). We found a similar pattern on arrays of volar whole dermis and volar whole tissue methylation arrays. Also published arrays on LMX1b knockdown limb bud skin also demonstrate suppression of SHOX (FIG. 13; mouse homologue) and EMX2 (FIG. 14). From our own microarray analysis of LMX1b knockdown fibroblasts at 24 hours, we also find early suppression of EMX2 (40%), and SHOX (30%) both ranking around the top 5% of most suppressed transcripts. Thus in a total of 3 distinct in vivo microarray studies, and 2 distinct knockdown array studies we find matching suppression of SHOX/EMX2 in conditions of volar tissue or knockdown LMX1b. These results again strongly suggest SHOX/EMX2 suppression might mediate LMX1b knockdown effects (FIG. 1).

Prophetic Investigation of the Transcription Factors and Cell Signaling Agents that Allow Fibroblasts to Induce Ectopic KRT9 in Non-Volar Keratinocytes.

Multiple lines of evidence support a role for PAX9 to promote a volar phenotype. However, no gain-of-function or post-natal role in adult limb fibroblasts has been tested. PAX9 is both overexpressed and knocked-down to test its sufficiency and necessity for KRT9 induction in the context of both normal volar fibroblasts and LMX1b knockdown fibroblasts.

Whether PAX9 upregulation is necessary and/or sufficient to endow non-volar fibroblasts the ability to induce KRT9 in non-volar keratinocytes is determined. We first concentrate on PAX9 knockdown, using routine techniques established in the lab (FIGS. 6-10). As in FIGS. 4, 6 and 9, volar and non-volar fibroblasts are expanded from extensive existing frozen stocks of 5 human donors. Knockdown using scrambled siRNA (Dharmacon D-001910-10-05) or PAX9 siRNA (L-012242-00-0005) is performed with nucleofection (Lonza 4-D) on 1 million fibroblasts. After plating for 1 day in a 10 cm² dish, 250,000 foreskin keratinocytes are added. Following 3 days, cells are harvested and tested for KRT9 induction. This experiment is repeated, replacing volar fibroblasts with knockdown LMX1b non-volar fibroblasts (for PAX9/LMX1b double knockdown).

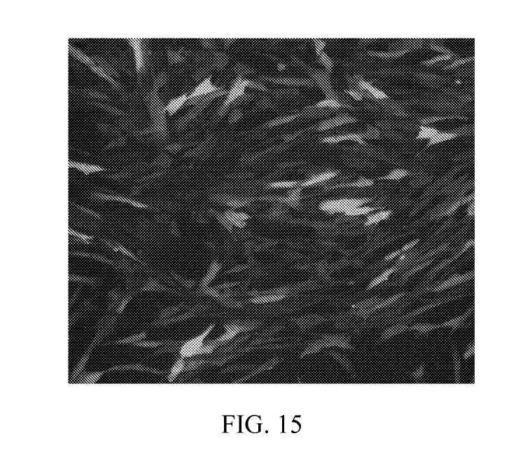

After testing the necessity of fibroblast PAX9 in KRT9 induction, its sufficiency is tested. As seen in FIG. 15, our lab has already performed transient transfections for the overexpression of GFP. We have already purchased PAX9 cDNA (Gene ID#5083)) and begun cloning into pcDNA3.1 vector. PAX9 or GFP is nucleofected into non-volar fibroblasts, and then incubated with foreskin keratinocytes and tested for KRT9.

In parallel, RT-PCR and western blotting are used to quantify the degree of over- or under-expression of manipulated gene. Successful results are verified with several distinct siRNA species and compared to scrambled controls. Finally, rescue studies are attempted where larger amounts of transfected transcripts should overcome the effects of limiting siRNA.

Based on our extensive microarrays demonstrating elevated PAX9 in volar fibroblasts, and increased PAX9 after LMX1b knockdown/KRT9 inductivity in non-volar fibroblasts, we anticipate that PAX9 overexpression in non-volar fibroblasts will endow them with the ability to induce KRT9. It is likely that PAX9 will also be necessary for induction and that knockdown of PAX9 will eliminate the capacity for volar fibroblasts or LMX1b knockdown non-volar fibroblasts to induce KRT9. If not the case, this raises the limitation that other homeobox transcription factors might compensate. For example, on several in vivo arrays we also see elevations of the homeobox LHX2 and LHX9 in volar fibroblasts—though these were not elevated in LMX1b knockdown fibroblasts which have the ability to induce KRT9. This highlights that the mechanisms of volar fibroblasts and non-volar LMX1b knockdown fibroblasts to induce KRT9 are not likely identical. Although we are focusing on overlapping features as clues to function, redundant mechanisms might exist in native volar fibroblasts which are absent in LMX1b knockdown fibroblasts. LHX2/9 might be examples. Thus, it is possible that PAX9 might be necessary for KRT9 induction in LMX1b knockdown non-volar fibroblasts, but perhaps not necessary in volar fibroblasts because of redundant pathways like LHX2/9. Therefore an alternative would be to then test double knockdowns of LHX2 or 9 and PAX9. Another alternative hypothesis might be that simultaneously transcripts which are upregulated by LMX1b (PAX9) and those downregulated (SHOX/EMX2) must be coordinately modified to stimulate KRT9. Another alternative effort would be knock-in of PAX9 and knock-down SHOX/EMX2 simultaneously.

Whether SHOX/EMX2 downregulation is sufficient and/or necessary to endow non-volar fibroblasts the ability to induce KRT9 in non-volar keratinocytes is determined. In these experiments, the transcription factors most downregulated in volar fibroblasts are investigated. Two transcription factors were together and highest as suppressed in volar fibroblasts: SHOX and EMX2. We found that SHOX/EMX2 is downregulated in the conserved mRNA signature of volar fibroblasts (Table 1). SHOX/EMX2 are downregulated by LMX1b knockdown in limb skin or in non-volar fibroblasts (30-40% at 24 hours; data not shown) which acquire the ability to induce ectopic KRT9. Both are downregulated in LMX1b knockout mice (FIGS. 13 and 14). Also, both SHOX and EMX2 have dorsum-limited expression in the developing limb and brain respectively. This suggests they might specify dorsal versus ventral fates in multiple tissues. Also, SHOX is mutated in some conditions of altered limb development, Langer Mesomelic Dysplasia (OMIM #249700). EMX2 has no known limb defects, but as for both genes, their postnatal function—in the skin or otherwise—has not been examined. We hypothesize that as for LMX1b, their continued suppression in volar structures maintains their ongoing identity. Therefore we predict that SHOX/EMX2 downregulation may mediate KRT9 inductivity and is downstream of LMX1b suppression.

Whether SHOX (Dharmacon L-011107-00-0005) or EMX2 (Dharmacon L-017904-00-0005) knockdown can endow non-volar fibroblasts the ability to induce KRT9 just as LMX1b knockdown does is tested first. Next, whether double knockdown of both EMX2 and SHOX is additive (compared to each separately) or necessary for KRT9 induction is then tested. After testing the sufficiency of knockdown to endow KRT9 inductivity, EMX2 (GFP tagged cDNA Origene RG228105), SHOX (GFP tagged cDNA Origene RG218605), and GFP plasmids (pCMV6-AC-GFP Origene PS100010) overexpressed in transient transfections to determine if downregulation is necessary for normal volar fibroblast induction of KRT9 expression. EMX2, SHOX or GFP plasmids with LMX1b siRNA are then overexpressed in non-volar fibroblasts to determine if these cells become resistant to the effects of LMX1b knockdown.

We anticipate that the specification of ventral fate will require the suppression of both EMX2 and SHOX simultaneously. While the LMX1b knockout mouse shows full duplication of ventral structures, this is not seen in EMX2 or SHOX knockout mice. Because both are downstream of LMX1b, and both suppressed in ventral tissue, we hypothesize that one might compensate for the other. We therefore also anticipate that overexpression of either will prevent LMX1b knockdown from endowing KRT9 inductivity in non-volar fibroblasts. Similarly, the overexpression of either will prevent volar fibroblasts from their normal ability to induce KRT9. These results will help define the genetic network which regulates volar skin identity, and add to evidence that this is an active process which maintains skin identity.

EMX2 and SHOX represent even still just a subset of downstream transcription factors from LMX1b. Although it is likely that EMX2 and SHOX are the dominant downstream factors given that they were the only ones to appear in both a conserved non-volar gene list (palm&sole) and an LMX1b knockdown gene list, others might exist. An alternative might then be to do CHIP arrays with LMX1b to determine a fourth array to define downstream genes. This would provide an even more robust bioinformatic platform to cross-reference biologically relevant genes. A minor limitation of this aim is the possibility that the GFP tag of EMX2 or SHOX might interfere with function. An alternative will be to use untagged EMX2 and SHOX cDNAs which are available. Alternatively, we will concentrate on direct signaling control by lmx1b on released factors such as Dkk-1. Other alternative areas for consideration include entirely non-genomic mechanisms of volar induction such as volar connective tissue and could involve evaluating the capacity for devitalized volar dermis to promote KRT9 induction after seeding with non-volar fibroblasts.

The sufficiency and necessity of DKK-1 release for ectopic KRT9 induction in keratinocyte cocultures with volar fibroblasts, lmx1b knockdown non-volar fibroblasts, and mutants. As described above, the transcription factors which control the volar identity of fibroblasts and the capacity to induce KRT9 are defined. However, for full mechanistic insight into the process of keratinocyte phenotype control by fibroblasts, we must also define the down-stream signaling agents. These agents might even eventually be useful as adjunctive therapies in addition to volar fibroblasts to induce ectopic volar skin. These experiments focus on DKK-1 which has already been proposed to inhibit melanocytes function in volar epidermis. Addition of DKK-1 to skin cultures also causes epidermal thickening; its necessity was not tested. Also, the sufficiency and necessity of DKK-1 to cause induce KRT9 has not been tested. Finally, the necessity of DKK-1 to mediate the capacity of LMX1b knockdown fibroblasts to induce KRT9 has also not been tested. Supporting its likely role, we find that DKK-1 is indeed increased after LMX1b knockdown, coincident with the ability to induce KRT9 (FIG. 8). Therefore the sufficiency and necessity of DKK-1 in scenarios of KRT9 induction is tested—first in normal volar fibroblasts, and if successful then in mutants.

First, sufficiency is tested by adding 0.03 μg/ml rDkk-1 (R&D 5439-DK) to foreskin keratinocyte cultures for 24 hours (based on co-culture conditions) and then measure for KRT9 induction. To test necessity, co-cultures are initiated as described above. Coincident with the addition of keratinocytes, 0.1 m/ml of neutralizing Dkk-1 antibody (R&D AF1096) is added, which has been shown to decrease Dkk-1 activity by 50%, with higher concentrations blocking >90%. After co-culturing, quantitative RT-PCR is performed as described above for KRT9. Volar fibroblasts are tested and, if successful, LMX1b knockdown fibroblasts. If PAX9 knock-ins or EMX2/SHOX knock-downs successfully induce KRT9, then these mutants are tested as well for the necessity of DKK-1.

We anticipate that DKK-1 will be sufficient for KRT9 induction, as has been shown for wnt5a. We also anticipate it will be partially required for KRT9 induction by volar fibroblasts, LMX1b knockdown fibroblasts, and possible PAX9/SHOX/EMX2 mutants. If our above hypotheses are not supported by experimental evidence, then other signaling agents must be considered. It has already been published that wnt5a is sufficient for KRT9 induction, and wnt5a was elevated on our arrays of volar fibroblasts (Table 1). Although we find it only minimally elevated after lmx1b knockdown, this is an alternative to consider. To identify other candidates, we have performed bioinformatics on our gene lists to identify those signaling agents which are elevated normally in volar fibroblasts and are also elevated in LMX1b knockdown. These candidates may be pursued as alternative approaches and include: IL-24 (top 98.8^{th} percentile), IL-12A (98.6^{th}) as well as less researched extracellular signalers such as SEMA3a (99.2^{th}) and TFPI2 (99.2^{th}).

Although LMX1b knockdown and potentially modulation of PAX9/SHOX/EMX2 might stimulate KRT9—and perhaps even requiring DKK1—it remains possible that a full volar phenotype might not be induced by single gene/protein manipulation, and even that the means of volar induction of LMX1b knockdown fibroblasts might represent a subset of the in vivo mechanism of normal volar induction. Supporting the possibility that a single agent like LMX1b knockdown can indeed fully recapitulate normal volar induction are multiple lines of evidence. Firstly, we find that both normal volar fibroblasts and LMX1b knockdown fibroblasts induce KRT9 to the same degree—by around 3-4 fold (FIGS. 4&10). Secondly, KRT9 is a suprabasal keratin and thus a late-stage marker of volar identity. Thirdly, it is likely that a limited set of extracellular signaling cues will define volar identity, and these will highly overlapping if not identical in both volar and LMX1b knockdown non-volar fibroblasts. Finally, the LMX1b knockout mouse shows full duplication of ventral structures which is the most convincing of all. However, since DKK-1/PAX9 upregulation and SHOX/EMX2 downregulation are downstream of LMX1b an equivalent argument cannot be made regarding fibroblasts with these agents manipulated. Therefore an alternative aim will be to define the transcriptional profile of “volarized” keratinocytes exposed to these cells and compare them to native volar keratinocytes. Similarly, a comparison of both in 3-D cultures (FIG. 5) could define any phenotypic differences. The results of this alternative aim would be defining whether partial “volarization” after manipulation of these volar factors occurs and support the further search for other signals.

If our results do not match those expected, then a second limitation is that in vivo signals might act differently from the proposed in vitro tests. Therefore, a second alternative would be to use mouse models such as crossing the available Col 1a2-cre-er mouse (JAX#016237) to floxed LMX1b to see how much adult tissue identity could be fully converted. We have acquired the floxed LMX1b mouse (Randy Johnson, M D Anderson; see letter of support) to prepare for this possibility. If these mice have volarization of the back skin for example, then we will have an in vivo system to define the signaling agents which are responsible using exogenous proteins or inhibiting antibodies.

Prophetic Investigation of Tissue Homeostatic Mechanisms which Maintain Non-Volar Skin Identity Using Both In Vitro and In Vivo Models.

Whether volar fibroblast induction of KRT9 is inhibited in the presence of non-volar fibroblasts is determined. To overcome any inhibition, the ideal ratio of added volar-fibroblasts to established non-volar fibroblast/keratinocytes for KRT9 induction in both in vitro and in vivo assays is defined.

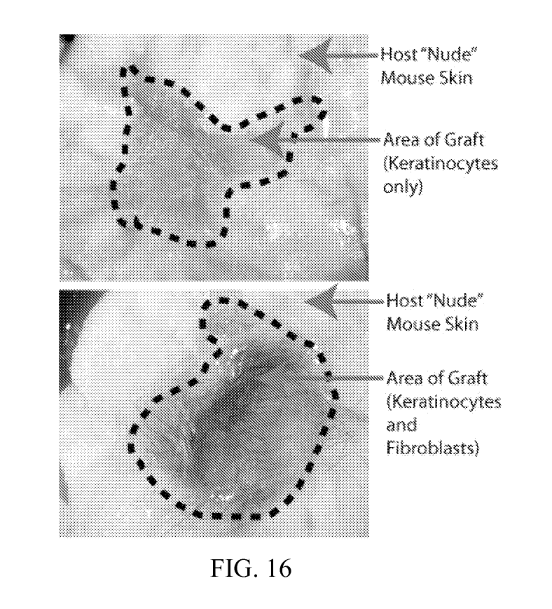

Tissue identity is remarkably static. Despite constant cellular turnover, even adjacent areas of skin such as at the transition of volar to non-volar maintain their identity. Therefore, it is likely the case that redundant mechanisms maintain tissue identity. These have been investigated at the genetic level with epigenetic mechanisms for example. However, to our knowledge, investigations on how multicellular tissue identity is maintained have not been performed. These questions will become increasingly important in regenerative medicine as cellular therapy is used. The present inventors address the hypothesis that in the case of volar tissue conversion, non-volar fibroblasts inhibit the ability of volar fibroblasts to induce ectopic KRT9 in keratinocytes. In the process, parameters for optimizing a human clinical trial are defined. Although 2-D cell culture system has the advantage of speed and reproducibility, in vivo mouse HAT assay (FIG. 16) experiments given their greater applicability to our eventual goal of human use are performed in parallel.

We have established that KRT9 is induced roughly 3-4 fold when 1 million volar fibroblasts are added to 250,000 non-volar keratinocytes. Here, co-cultures of increasing amounts of non-volar fibroblasts with 250,000 non-volar keratinocytes are first established. One million volar fibroblasts are then added to determine the degree to which KRT9 induction is inhibited (from 3-4 fold average). We then determine at which ratio of volar:non-volar fibroblasts KRT9 inhibition is minimized. Whether conditioned media from non-volar fibroblast mediates such an inhibitory effect is also tested, given that fibroblasts have little cell-to-cell contact. For in vivo studies, analogous studies in the HAT assay (FIG. 16) are performed. The present inventors have experience with reconstituting full hair follicles from cell slurries, both in subcutaneous injections and in the Lichti hat model (FIG. 16). Here, human non-volar fibroblasts (10 million) and keratinocytes (10 million) are combined to reconstitute non-volar skin in the 1 cm diameter silicon “hat” chamber on a SCID mouse. After graft take (4 weeks), sub-epidermal injections of 0.1, 1 and 10 million volar fibroblasts are performed, then epidermis thickness is measured using non-invasive confocal scanning laser microscopy as we do routinely on mice. We estimate thickening to occur at week 3-4 given typical reconstitution timelines (FIG. 5,). The graft is then excised and epidermal thickness is measured by histology and KRT9 levels by qRT-PCR to define which ratio optimally “volarizes” skin.

We anticipate that non-volar fibroblasts will inhibit KRT9 induction by volar fibroblasts. Evidence for success are the ligands which have been associated with inducing the volar phenotype and their functions. For example, wnt5a is published to induce KRT9. Wnt5a activates β-catenin independent pathways. Similarly, we have confirmed that wnt-inhibitory protein DKK-1 is elevated in volar tissues (FIG. 8) and it has been shown to decrease melanocytes function in volar skin. Given the role of canonical wnts in promoting hair follicles morphogenesis and cycling, the absence of hair follicles in volar skin, and the upregulation of wnt5a/Dkk-1, volar skin could be a product of a canonical wnt-poor environment. Therefore the presence of wnts in non-volar fibroblasts will likely inhibit KRT9 induction. Determining the exact degree of this inhibition is important for measuring an ideal elevated ratio of volar fibroblasts necessary to overcome this inhibition. This ratio can then be directly used to determine the quantity of volar fibroblasts to inject based on the known density of non-volar fibroblasts.

We anticipate that this negative effect of non-volar fibroblasts may be present in the conditioned media from non-volar fibroblasts. The evidence for this is that there is little physical contact of fibroblasts and keratinocytes in epithelial mesenchymal interactions in the skin given the extensive basement membrane. Therefore modulation of KRT9 expression likely occurs exclusively through paracrine signaling molecules.

Limitations are the possibility that no inhibition of KRT9 induction will occur from non-volar fibroblasts. If this is the case, then this bodes well for this therapy. Another limitation is the relative difficulty of in vivo animal experiments; an alternative would be using 3-D systems (FIG. 5). A final limitation is that while we will not define the specific factors which inhibit KRT9 inductivity. An alternative would be to fractionate the media using protein size exclusion columns followed by mass spectrometry screening of responsible fractions, but this is beyond the scope of the grant.

Whether minority volar fibroblast have decreased survival, proliferation or gene expression in majority non-volar fibroblast cultures is then determined. Whether fractionated dosing with serial injections of volar fibroblasts to non-volar fibroblasts/keratinocytes may overcome this indirect effect and enhance KRT9 induction in both in vitro and in vivo assays is defined.

As mentioned above, multiple mechanisms likely maintain tissue identity. For example, circulating fibroblasts or migrating fibroblasts during wounding likely die or adopt the phenotype of their surrounding cells at their destination. However, dermal fibroblasts default is to maintain their identity even after removal from the body. Previous work demonstrated that dermal fibroblasts maintain their hox signature in culture. Our results demonstrating a conserved volar phenotype corroborate that group's findings (Table 1). This is a paradox: if fibroblasts by default maintain their positional memory, how is this memory lost in the cases of ectopic fibroblasts such as would happen with migrating ectopic fibroblasts during wounding or hematogenously seeded ectopic fibroblasts? Overcoming these mechanisms will be important for the ectopic injection of fibroblasts to induce KRT 9. We hypothesize that cell signaling occurs in majority populations of fibroblasts to maintain their identity; minority populations will show decreased proliferation, survival or native gene expression. The rationale is to test this hypothesis in vitro and in vivo.

Our first effort will be to quantitate survival, proliferation and gene expression in minority site-mismatched fibroblasts compared to the majority population in fibroblast co-culture experiments. Established commercially available techniques for cellular labeling are used prior to placement in co-cultures. Specifically, Cell Trace CFSE (green) and Cell Trace Violet cell labeling are employed which are stable up to 10 generations of cells, not transferred to adjacent cells, and can be distinguished by flow cytometry. These approaches have been validated and published, mostly for T cell activation studies, but also for adherent cells. Proliferation and necrosis are quantitated by analytical flow cytometry of each distinct population. Cell sorting is also utilized for analyzing gene expression by RT-PCR.

We will first perform positive controls using both green and violet dyes in a homogenous population. Specifically, we will label a subset of volar cells with green, and a larger population of volar cells with violet. We will verify that these differently labeled groups in a homogenous population have identical rates of proliferation, cell death, and gene expression. We will do the same with non-volar cells. For both we will use serum starved and serum added fibroblasts as positive controls to demonstrate differences in proliferation. After confirming these controls, we will label minority populations of volar and non-volar populations in majority non-volar and volar populations respectively. We will use analytical flow to determine up to 10 population doublings, and measure for differences in proliferation or necrosis (DAPI exclusion, LSRIIb with UV laser) in minority populations. We will sort minority and majority populations for RNA extraction and qRT-PCR to see for example if PAX9 expression decreases and LMX1b expression increases in minority volar populations—so they match surrounding fibroblasts over time.

We will determine the length of time required for an effect of non-volar fibroblasts to inhibit the activities of volar fibroblasts. Then we will test if serial injections before this elapsed interval of volar fibroblasts into non-volar fibroblast/keratinocyte cocultures will increase KRT9 induction.

In parallel we will test with animal models. We will order inbred sex and strain (HLA matched; FVB) mice, one with a constitutive luciferase gene as a transgenic (JAX#008450). Fibroblasts will be cultured from different areas of the transgenic mouse (hind-limb, back, paw & abdomen), expanded to 1 million fibroblasts and either injected with identical numbers homo-topically (same place) or hetero-topically (mismatched location) in the HLA-matched non-transgenic host. Graft survival will be measured by whole-mouse non-invasive IVIS luciferase imaging (available in our animal facility) over the course of 4 weeks. We will quantitate if heterotopic grafts have decreased survival compared to homotopic grafts.

Anticipated Results, Limitations and Alternatives:

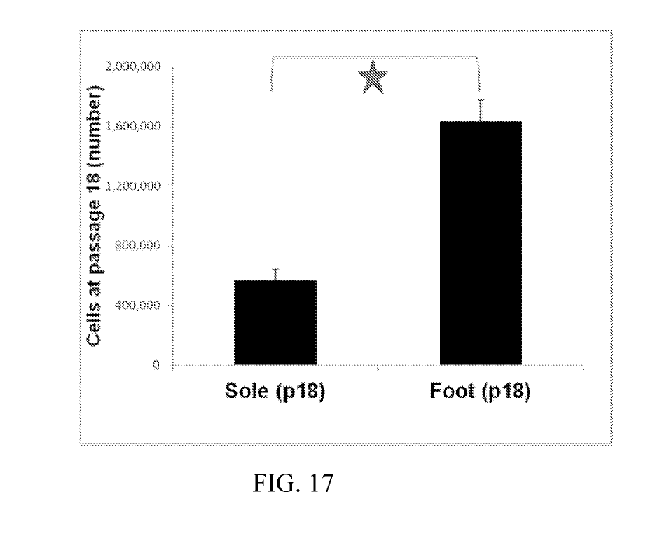

Given every tissue's fixed identity despite injury and constant cellular turnover, we predict tissue-level homeostatic mechanisms maintain tissue identity. (If this is not the case, see limitations below). We clearly demonstrate that volar and non-volar fibroblasts maintain very distinct mRNA expression signatures in culture (Table 1). These lead to phenotypic differences which we have also noted, for example that sole fibroblasts proliferate more slowly at late passage (FIG. 17). However, we have not tested the effect of mixed populations. We anticipate that in the same way that cultured cells release unidentified autocrine factors which require that they be plated at minimum densities, in vivo site specific fibroblasts will be homotrophic.

Beyond this theoretical evidence for the inhibition of ectopic fibroblasts, practical experience in the use of autologous fibroblasts for the treatment of wrinkles also supports these mechanisms. These trials all consisted of serial injections of mismatched fibroblasts, and serial injections are recommended also for clinical use. If ectopic fibroblasts survived equally, no repeat injections would be required—but they are. We hypothesize that native fibroblasts will repress ectopic site-mismatched fibroblasts. The importance of this aim is creating an in vitro system to demonstrate this effect as a prelude to dissecting its mechanism.

We anticipate that in the presence of majority non-volar fibroblasts, minority volar fibroblasts—compared to their status in homogenous solo-cultures—will demonstrate some combination of decreased proliferation, increased cell death, decreased PAX9, and increased LMX1b, SHOX or EMX2. Given the 3 day timeframe for KRT9 induction, we anticipate this effect to occur after 48 hours, and that divided serial injections every 48 hours will be superior to an equal total amount given once.

The main limitation of this aim is the possibility that there is no inhibition of minority volar fibroblast populations in majority non-volar fibroblast co-cultures. While this seems unlikely, this negative result will itself be illuminating and bode well for Aim 3. Another limitation (also applicable to Aim 2c) is the relative difficulty of in vivo animal experiments; an alternative would be using 3-D systems (FIG. 5). A limitation of the in vitro effort is the possibility that other tissue components such as extracellular matrix, keratinocytes, or some other element not present in these fibroblast co-cultures might maintain non-volar tissue identity; therefore we will also test an in vivo model in parallel.

2c: Determine if wounding augments KRT9 induction in cultures of non-volar keratinocytes with variable ratios of volar to non-volar fibroblasts using both in vitro and in vivo models. Test whether adult versus neonatal keratinocytes have similar potential for KRT9 induction.

Rationale:

Methods of tissue conversion exist in mice. As illustrated in FIG. 16 adding appropriate cell slurries to chambers allows the reconstitution of hair follicles. Trauma converts resting telogen hair follicles to enter the anagen growing phase. Also in mice, large excisional wounding allows complete reprogramming and a de novo recapitulation of embryogenesis for organogenesis of a new hair follicle in the center of a scar. In humans, laser therapies to treat photoaging also are hypothesized to “reset” tissue function by virtue of wounding. In fact, the more ablative lasers are the more successful therapies. All of these phenomena capitalize on a wounding environment. Therefore we hypothesize that wounding might allow a window for identity conversion and actually mitigate any direct (Aim 2a) or indirect (Aim 2b) inhibition on KRT9 induction by native non-volar fibroblasts upon volar fibroblast addition.

A second rationale for this aim is to test adult versus neonatal keratinocytes for their potential to induce KRT9. Although for the preliminary data in this grant we have demonstrated multiple instances of KRT9 induction of foreskin keratinocytes (FIGS. 4,6,9&10), we ourselves have not tested adult keratinocytes. There is a large body of evidence to predict that adult keratinocytes would have equivalent inductions to neonatal keratinocytes. Yamaguchi et al demonstrated that when transplanted to volar dermis, adult non-volar express abundant KRT9 (See Introduction to Resubmission; FIG. 0;), which his group repeated in a separate study. My work and others have shown the ability of adult keratinocytes to even convert to sebocytes with hair inductive fibroblasts. Nevertheless, this will be an important variable to test.

Approach:

In aim 2a we will optimize ideal ratios of added volar fibroblasts to existing non-volar fibroblast/keratinocyte co-cultures. In this aim, we will establish non-volar fibroblast/keratinocytes co-cultures. We will then perform scratch injuries on select plates, and then add a sub-optimal amount of volar fibroblasts which in Aim 2a were found to only partially induce KRT9. We will test if KRT9 induction is augmented.

In aim 2b we will define ideal divided dosing for serial applications of ectopic volar fibroblasts to existing non-volar/keratinocyte co-cultures. Analogous to above, we will test if serial scratching before suboptimal divided dosing of ectopic volar fibroblasts will enhance KRT9 induction.

Also, as in Aim 2a, we will use the HAT assay (FIG. 16) to create non-volar human epidermis. The only difference from those proposed experiments will be that we will perform needle punctures in the skin before the subepidermal injection of sub-optimal number of volar fibroblasts to test for enhanced KRT9.

Finally, we will add volar fibroblasts to either adult or neonatal foreskins to test for KRT9 induction.

Anticipated Results, Limitations and Alternatives:

We expect that in culture or HAT in vivo assay wounding will enhance KRT9 induction. If not the case, then we will consider alternate wounding methods. For example, our group has extensive experience with the wound-induced hair neogenesis model. Rather than hair neogenesis, would volar skin reconstitution occur if volar fibroblasts were injected into the wound? The main limitation of this aim is the possibility that wounding will not enhance engraftment. Other alternative methods to enhance engraftment might be pre-treating volar fibroblasts with growth factors, but this would considerably complicate the regulatory path for testing in human subjects. Other factors to test would be volar ECM proteins.

We predict that adult and neonatal keratinocytes will have equal potential to induce KRT9 in vitro. If this is not the case, this could be an artifact of an in-vitro system given in vivo evidence for adult ectopic KRT9 expression. An alternative would be testing in vivo as above for the HAT assay. If actually the case that adult keratinocytes have inhibited capacity for induction of KRT9, then we may test methods such as wounding as above to enhance that capacity.

Prophetic Clinical Trial for Ability of Autologous Human Volar Fibroblasts to Induce Volar Epidermal Gene Expression.

Cellular therapy holds great promise in regenerative medicine but is present in very rudimentary forms. Allogeneic products such as living keratinocyte/fibroblast bilayered grafts (Apligraf,) or living fibroblast only grafts (Dermagraft) are used as wound therapies. Recently autologous fibroblasts have been FDA approved and are marketed for treating wrinkles as an injectable filler (Fibrocell). Despite this considerable clinical effort, an optimization of cellular engraftment in humans has not been accomplished to our knowledge. Partially this is because none of these indications have clear quantitative molecular endpoints for optimization.

The central significance for this Example is that KRT9 and the conversion to volar skin present an ideal test case to begin answering basic clinic questions regarding efficacy of cellular therapy. The present inventors will biopsy volar and non-volar skin, expand fibroblasts in the clinically certified Hopkins Cellular Therapy Core, and verify in vitro ability to induce KRT9 ectopically for purposes of an FDA Investigational New Drug Application (IND).

All cellular clinical work will be done in the Hopkins Cellular Therapy Core which has complete regulatory approval by governing organizations (CLIA, FACT, AABB & CAP). The practical issues regarding the clinical use of autologous cells include defining exact variables regarding cellular expansion: total number of fibroblasts expanded in 4 weeks, verification of purity, and verification of efficacy in standard clinical production. For expansion, it will be preferable to avoid the 10% FBS formulations we have been using to decrease future costs and potential scarcities in the FBS market with market demand from expanding mesenchymal stem cell industry efforts. Therefore we will test 0% or 2% supplemented “serum-free” fibroblast media such as Millipore FibroGRO(SCMF001) and Lonza FBM/FGM-2(CCO3132). The FDA has commented that oncogenicity tests are not necessary given the minimal manipulation of cells during expansion. Verification of purity will be done by staining with vimentin. We will also compare nonvolar to volar fibroblasts for the presence of CD24. CD24 was the 87^{th} highest transcript enriched in volar fibroblasts (out of 46,878; Table 1). This might be a further candidate for quantitation of purity of volar fibroblasts. For eventual clinical use, volar cells will be expanded in the Hopkins Cellular Therapy Core. In certain embodiments, for ease of eventual clinical use, frozen cells after GMP expansion are tested for efficacy. We have already tested that thawed cells are effective in our research laboratory (FIGS. 4, 6& 9), but have not done so in a GMP context. Therefore we will assess if recently thawed volar fibroblasts also have the capacity to induce KRT9.

We predict that both the Millipore and Lonza medias will allow for equivalent KRT9 induction. Given our culture conditions, we expect the purity to be above 90% for vimentin positive cells. We predict CD24 to be slightly but not exclusively elevated in volar tissue.

After FDA and IRB approval, the present inventors inject autologous volar and nonvolar fibroblasts in paired areas of the buttocks. The injected cells and overlying epidermis are removed through skin biopsy after noninvasive imaging demonstrates thickened epidermis/stratum corneum. For safety reasons, it is ideal to remove the entire injected area after a change of phenotype is identified.

Volar (sole) and non-volar (foot) 3 mm punch biopsies will be taken from consented subjects. Fibroblasts will be expanded from these tissues as we have done (FIGS. 4-13), but with low FBS media formulations. If frozen cells are found to still be efficacious in inducing KRT9 expression, then aliquots of cells will be frozen in standard clinical cell freezing media (2.5% Human serum albumin, 5% DMSO, 6% Hetastarch in sterile saline). Each aliquot frozen will equal a single dose. In particular embodiments, total volumes will be 700 μl—the limit for a subepidermal injection without escape of cells from a concentrated 5 mm diameter “bleb” in the skin in the experience of the PI during routine clinical lidocaine injections and the cellular therapy core in almost 10 live-cell sub-epidermal cancer vaccine immunization trials. Total number of cells may vary. In some embodiments, the absolute upper limit of cells will be 30 million volar cells. In certain embodiments, we predict the number will be 10 million based on our in vitro ratios of keratinocytes to fibroblasts.

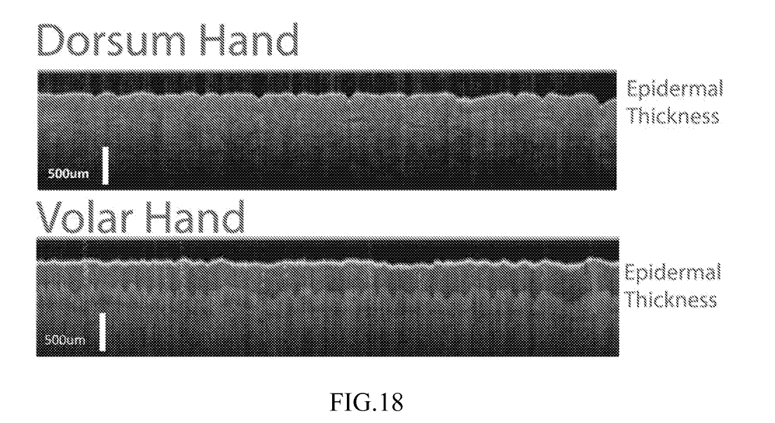

Frozen cells will be washed with PBS. We will perform 3 injected areas on the left side (volar) and 3 areas on the right side (non-volar) of the buttocks. Injection sites will be carefully marked, measured and photographed with and without transparency paper used to label landmarks (moles, hemangiomas, sacro-iliac joint, cleft of buttocks i.e.) and injection sites. After 2 weeks, we will perform weekly then monthly monitoring of epidermal thickness with optical coherence tomography (OCT) which we have tested on human subjects to verify its feasibility (FIG. 18,). We will quantitate epidermal thickness. Once thickness shows an increase we will perform a 6 mm punch to remove the entire injected area in ½ of the subjects. The remainder of subjects will be monitored for a period of 6 months to determine the longevity of the effect of increasing epidermal thickness with biopsy at the completion of the 6 month period.

We imagine some leakage of fibroblasts will occur during injection, but since we will inject 20% more cells than considered necessary, this should not be itself an issue. We predict easily identifying injection sites given our multiple analogous and successful clinical studies in the Hopkins Dermatology Clinical Translational Unit. Alternatives will be to attempt dose escalation studies with higher and lower concentrations, and divided dosing studies. Further alternatives will be to test whether injury such as surface dermabrasion or ablative laser enhance the effects of injected cells. A completely alternative hypothesis is that connective tissue materials from volar dermis might enhance volar induction; seeding non-volar fibroblasts and keratinocytes on devitalized volar dermis versus non-volar dermis is one means to test this hypothesis.

Epidermis analyzed by both histology and RT-PCR for features of volar phenotype (KRT9, epidermal thickness, stratum corneum thickness, presence of PAX9^{high}, LMX1b^{low}, EMX2^{low}, SHOX^{low} fibroblasts. Although phenotyping of volar injected areas might show gross changes such as greater epidermal thickness than in non-volar injected areas, more subtle molecular changes might occur which will require careful testing. We capitalize on the verification of the importance of these transcription factors to use them as markers to follow ectopic fibroblast presence and persistence.

The 6 mm punches are split into two halves. One half is sent for paraffin embedding and staining for H&E as well as KRT9. The other half is homogenized for qRT-PCR. KRT9, PAX9, LMX1b, EMX2, SHOX and other markers as defined in epidermal microarrays are assayed.

We anticipate that as we see in vitro, volar cells increase KRT9 mRNA and epidermal thickness, though not to in vivo levels. We expect positive results that PAX9 is increased in volar injected sites, and likewise EMX2, SHOX and LMX1b is lower. We suspect that full conversion for a volar phenotype requires greater pressure and friction as seen in the palms and soles.

Limitations of this aim are the possibility that post 6 month time points show greater changes. An alternative would be a late time course study which. Another limitation is that pressure—with volar fibroblasts—might be an important factor to fully induce KRT9; amputee testing (see below) might in fact show better results than these initial trials, though this is a vulnerable population.

Application to Amputees:

Creating ectopic volar skin at the stump site of amputees is described herein. An optimal dose and frequency is defined with in vitro and animal models, and tested in a proof-of-concept study in healthy individuals the ability to convert non-volar to volar skin. The results from above will verify the markers to test for the persistence of volar fibroblasts in amputees. Results also from above will inform exact dosage and frequency to test in future trials. The results above also identify the degree and longevity of response which will directly inform the design of future trials. We propose at the completion of this grant will be optimizing again in healthy subjects the ideal dose and frequency of cellular delivery in a more formal Phase ½ human trial (here optimized only in animal and in vitro models). Only after completion of this second healthy subject study will we attempt a trial on the vulnerable population of amputees. In that trial (Phase ⅔) we will scale up the number and volume of cells to inject according to the increased area of the stump. We predict that this will be feasible given the large increases of cell numbers at later doublings of fibroblast cultures and the use of ring-blocks for anesthesia in an out-patient setting. Importantly we will focus later trials on measuring for increased usability of prosthetics.

1. A method for altering skin identity in a patient comprising the step of transplanting autologous fibroblasts into the target skin site of a patient, wherein the autologous fibroblasts are obtained via a skin biopsy from the desired skin type site.

2. The method of claim 1, wherein the target skin site is the stump site skin of an amputee and the desired skin type site is volar skin of the amputee.

3. The method of claim 1, wherein the target skin site is an alopecia site on the patient and the desired skin type site is haired scalp.

4. The method of claim 1, wherein the target skin site is a scar and the desired skin type site is from an area adjacent to or surrounding the scar.

5. The method of claim 1, wherein the target skin site is discolored skin and the desired skin type is from an area adjacent to or surrounding the discolored skin.

6. The method of claim 5, wherein the discolored skin is a port wine stain.

7. The method of claim 1, wherein the target skin site is a mismatched split thickness skin graft or other autologous skin graft and the desired skin type is from the contralateral skin of desired identity.

8. The method of claim 1, wherein the target skin site is a site with a predilection for a rash or ulcer and the desired skin type is normally resistant to that rash or ulcer.

9. The method of claim 8, wherein the site with a predilection for a rash or ulcer is a pressure ulcer of the sacrum and the desired skin type that is normally resistant to that rash or ulcer is volar skin.

10. A method for altering skin identify in a patient comprising the steps of:
a. obtaining a tissue sample from the desired skin type site of the patient;
b. culturing the tissue to expand the fibroblasts; and
c. transplanting the expanded autologous fibroblasts into the target skin site of the patient.

11. The method of claim 10, wherein the target skin site is the stump site skin of an amputee and the desired skin type site is volar skin of the amputee.

12. The method of claim 10, wherein the target skin site is an alopecia site on the patient and the desired skin type site is haired scalp.

13. The method of claim 10, wherein the target skin site is a scar and the desired skin type site is from an area adjacent to or surrounding the scar.

14. The method of claim 10, wherein the target skin site is discolored skin and the desired skin type is from an area adjacent to or surrounding the discolored skin.

15. The method of claim 14, wherein the discolored skin is a port wine stain.

16. A method for inducing volar skin at the stump site of amputees comprising the step of transplanting autologous volar fibroblasts to the non-volar stump site of the amputee.

17. The method of claim 16, wherein the transplantation step comprises injection of the autologous volar fibroblasts.

18. A method for inducing volar skin at the stump site of amputees comprising the steps of:
a. obtaining a volar skin biopsy from the amputee;
b. culturing the biopsy to expand the fibroblasts; and
c. transplanting the expanded autologous fibroblasts into the stump site of the amputee.

19. A method for treating alopecia in a patient comprising the step of transplanting autologous fibroblasts into the alopecia site of the patient, wherein the autologous fibroblasts are obtained via a skin biopsy from the haired scalp of the patient.

20. A method for treating alopecia in a patient comprising the steps of:
a. obtaining a tissue sample from the haired scalp of the patient;
b. culturing the tissue to expand the fibroblasts; and
c. transplanting the expanded autologous fibroblasts into the alopecia site of the patient.

21. A method for altering the skin identity of a scar in a patient comprising the step of transplanting autologous fibroblasts into the scar site of the patient, wherein the autologous fibroblasts are obtained via a skin biopsy from a skin site adjacent to or surrounding the scar site of the patient.

22. A method for altering the skin identity of a scar in a patient comprising the steps of:
a. obtaining a tissue sample from a skin site adjacent to or surrounding the scar site of the patient;
b. culturing the tissue to expand the fibroblasts; and
c. transplanting the expanded autologous fibroblasts into the scar site of the patient.

23-25. (canceled)
